(19) United States
(12) Patent Application Publication
(10) Pub. No.: US 2022/0354460 A1
(43) Pub. Date: Nov. 10, 2022

(54) EVENT-BASED ULTRASOUND SYSTEM

(71) Applicant: ACOUSTIIC INC., Pacific Palisades, CA (US)

(72) Inventors: Sean Taffler, Pacific Palisades, CA (US); Paul Reynolds, Renton, WA (US)

(73) Assignee: ACOUSTIIC INC., Pacific Palisades, CA (US)

(21) Appl. No.: 17/737,377

(22) Filed: May 5, 2022

Related U.S. Application Data
(60) Provisional application No. 63/184,417, filed on May 5, 2021.

(51) Int. Cl. A61B 8/00; A61B 8/08; G01S 15/89

(52) U.S. Cl. CPC A61B 8/4488; A61B 8/5207; A61B 8/56; G01S 15/8915

(57) ABSTRACT

Systems and techniques are provided for an event-based ultrasound system. An event-based ultrasound system may include a transducer array, a computing and imaging device, and a connection. The transducer array may include transducer elements and a timing system. The transducer elements may be connected to event-detection circuits. The event-detection circuits may generate events based on the electrical signals of the transducer elements. Data for an event may include an up-down value, a timestamp based on the timing system, and an address of a transducer element. The connection may connect the transducer array to the computing and imaging device. Data for the events generated by the event-detection circuits may be transmitted to the computing and imaging device across the connection.

### BACKGROUND

Each receive element of an ultrasound system may convert an incident ultrasonic wave to electrical energy that may be output from the receive element as an electrical signal. The electrical energy from a receive element may vary with time proportional to the frequency content of the incident ultrasonic wave. The electrical signal may be sampled to convert it into a form suitable for digital processing. The sampling may be done at a frequency that is greater than twice the incident frequency, which may be the Nyquist frequency. If the incident frequency is 1 MHz then the sampling frequency may be a minimum of 2 MHz and may typically be higher so that more data points on the wave can be captured. Higher sampling frequencies may result in a greater rate of data transfer and an increase in overall system complexity. Sampling at the Nyquist frequency may result in only two points being sampled per wave cycle. This may be mathematically satisfactory in a continuous wave environment, however imaging ultrasound may typically be performed using short bursts of ultrasound energy, so the sampling frequencies used in ultrasound systems may be higher than the Nyquist frequency. Anomalies may be introduced in reconstructed waveforms if they are sampled at the Nyquist frequency. Sampling a waveform assigns a value to the amplitude of the waveform. The value may be represented as a multi-bit binary number. The number of bits used to represent the value may be proportional to the desired precision of the amplitude representation. The number of bits used to represent a sample value may generally be in the range of 8 to 14 bits, which may correspond to from 256 to 16384 levels or steps. Sampling of an analog waveform to generate a digital representation of the waveform may be referred to as digitization or quantization.

Sampling a single transducer element operating at an ultrasound frequency of 1 MHz at the Nyquist frequency would correspond to a data rate of between 16 Megabits per second (Mbps) at the low end of resolution, and up to 28 Mbps. In many ultrasound systems used in medical environments, digitization is performed in the cart, and not the handpiece that includes the transducer elements and is closer to the patient. Current ultrasound systems may have more than 100 transducer elements, which may in turn generate 100 channels of analog electrical signals. This may result in a cable that connects the hand piece of the ultrasound system to the cart becoming unwieldy, as it may need to be accommodate wiring to carry 100 channels in parallel. The electrical signals carried across the cable may be degraded due to the interference of the neighboring electrical signals, length of cable, and electrical impedance mismatch, and the overall ergonomics and cost of the ultrasound system may be traded-off against imaging performance of the ultrasound system. When imaging takes place within the body of the patient, such as via catheters or endoscopes, the number and quality of connections in the cable may be limited by both size and curvature of artery, vein, or other channel in the body. For a transducer array with more than around 100 to 200 transducer elements, the number of signals to be transmitted may become difficult to manage outside of a static lab environment, or without the use of multiplexing which may reduce the performance of the system.

Instead of sending the analog electrical signal from a transducer element across a wire to be digitized at a remote digitizer, the electrical signal can be digitized closer to the transducer element. This approach may be limited by the available space near the transducer element as well as by heat and power consumption. Systems with low numbers of transducer elements record data constantly from every transducer element, which results in the recording of mostly zeros. For larger transducers arrays, such as those with thousands of transducer elements, if it were possible to digitize the signals close to the transducer elements using conventional digitization, the data rate for a transducer array with 1000 transducer elements may be between 16 Gigabits per second (Gbps) and 28 Gbps. This may also result in significant levels of power consumption, possibly hundreds of Watts, which may cause difficulties both in supplying enough power and providing appropriate cooling, especially given regulatory limits in a patient contacting device. To avoid these issues, current ultrasound systems that have transducer arrays with a large number of elements may use micro-beamforming methods to group receive responses from multiple transducers elements, often from 9 to 36 elements, into a single channel. However, doing this may degrade the quality of the image generated by the ultrasound system.

A further metric of imaging system quality is the signal to noise ratio of the detected signals. Typically, imaging systems, including ultrasound imaging systems, operate at about 80 db of signal to noise ratio. Long wire paths between a handset that includes the transducer elements and a cart used to process the signals from the transducer elements can severely degrade the signal to noise ratio in an imaging system. Making the wires shorter or digitizing the signal from the transducer elements closer to the transducer elements can ameliorate this.

### BRIEF DESCRIPTION OF THE DRAWINGS

The accompanying drawings, which are included to provide a further understanding of the disclosed subject matter, are incorporated in and constitute a part of this specification. The drawings also illustrate implementations of the disclosed subject matter and together with the detailed description serve to explain the principles of implementations of the disclosed subject matter. No attempt is made to show structural details in more detail than may be necessary for a fundamental understanding of the disclosed subject matter and various ways in which it may be practiced.

FIG. 1 shows an example representation of a circuit for an event-based ultrasound system.

FIG. 2 shows an example system for an event-based ultrasound system.

FIG. 3A shows an example arrangement for an event-based ultrasound system.

FIG. 3B shows an example arrangement for an event-based ultrasound system.

FIG. 3C shows an example arrangement for an event-based ultrasound system.

FIG. 4A shows an example arrangement for an event-based ultrasound system.

FIG. 4B shows an example arrangement for an event-based ultrasound system.

FIG. 5 shows an example procedure for an event-based ultrasound system.

FIG. 6 shows an example representation of data collection by an ultrasound system.

FIG. 7 shows an example representation of data collection by an ultrasound system.

FIG. 8 shows a computer according to an embodiment of the disclosed subject matter.

FIG. 9 shows a network configuration according to an embodiment of the disclosed subject matter.

### DETAILED DESCRIPTION

An event-based ultrasound system may allow for only relevant data from the transducer elements of an ultrasound system to be captured and recorded. This may allow images that otherwise could not be sent in real time to be transmitted, enabling real time responses such as robotic surgery, or may allow the images to be transmitted from bandwidth constrained locations such as battlefields or in-flight planes. An event-based ultrasound system may allow for a greater number of transducer elements to send data to any given transmission medium, increasing the maximum number of transducer elements that may be practically used in any application, such as in distributed sensor arrays, tomography, large volume imaging, or any other high element count system. In addition, an event based ultrasound system may enable the production of high quality imaging systems that have small form factors, reduced weight, and lengthened battery life due to the simplified demands of analog to digital conversion and bandwidth requirements, potentially allowing for wireless transmission of data from the transducer elements. In a large array, such as one with 1000 or more transducer elements, the threshold amplitude for capture of the signal may be varied to allow speckle imaging when it may be otherwise be prohibitive based on the necessary bandwidth or storage to perform full matrix capture of the array. Microbeamforming may not be required, and high element count arrays of any form may be realized. Event based capture may also be used with microbeamforming to construct these ultrasound arrays.

An event-based ultrasound system may use a single bit quantizer and timing system. The single-bit quantizer may be used to quantize electrical signals generated by transducer elements of the transducer array of the ultrasound system into a single bit, which may represent up-down values for the electrical signals. The timing system may synchronize the time stamping of data across the transducer array and may be used in the generation of timestamps for the up-down values generated by the single bit quantizer to indicate the time the event the up-down value was generated for occurred. Each up-down value may also have an associated address, which may indicate which transducer element of the transducer array generated the electrical signal that was quantized into the up-down value. A buffer may store and forward these addressed and timestamped up-down values to a host processor. The host processor may then generate the ultrasound image from addressed and timestamped up-down values.

Event-based ultrasound systems may only store and timestamp up-down values on changes in the up-down value for an individual transducer element. For example, the up-down value for a transducer element may initially be “0”, which may be addressed, timestamped, and stored in the buffer for sending to the host processor. The up-down value for that transducer element may not be addressed, timestamped, and stored in the buffer again until the up-down value has changed to “1”, which may occur when there is an up-edge in the electrical signal generated by the transducer element. Similarly, after the up-down value for a transducer element has changed to “1”, the up-down value may not be addressed, timestamped, and stored again until the up-down value changes back to “0”, which may occur when there is a down-edge in the electrical signal generated by the transducer element. The changes in the up-down value for a transducer element may be changes between +ve or −ve, Changes in the up-down value for a transducer element, representing changes in the electrical signal being output to the quantizer by the transducer element, may be subject to a threshold value in either direction. The threshold value may be dynamic and may be considered a memory for the last “value” or “values” of intensity at the transducer element. Noise may be ameliorated via hysteresis in the threshold values with larger gaps between upper and lower threshold values decreasing noise at the expense of sensitivity to slight changes in intensity. Each stored up-down value based on a change in the electrical signal output by a transducer element may be representative of an event for the transducer element. The data for an event may include the up-down value, for example, as a single bit and a timestamp indicating when the event occurred. The data for an event may also include the address for the transducer element, although some implementations may initially store data for the event without the address, for example, when an event for a transducer element is stored in memory that is local to, and specific to, the transducer element.

The conversion of the electrical signals generated by the transducer elements may be asynchronous, as the sampling frequency may be as fast as the underlying electronics can threshold and timestamp the data. With fast changing signals, this may occur at a frequency that is many multiples of the fundamental frequency. With a slower changing signal, the conversion may be just as fast, but the number of events recorded in the form of stored up-down values may be lower.

Data generated by the transducer elements of an ultrasound system may be considered bursty, as a series of pulses sent out by the ultrasound system, and an echo of those pulses may return to the transducer elements. Not all transducer elements may receive an echo of any of the pulses at the same time, and therefore not all transducer elements may generate an electrical signal caused by the receipt of an echo of a pulse at the same time.

An ultrasound system may, to allow for transducer dynamics, send out around five cycles of the ultrasound frequency as an ultrasound pulse. It may then be possible to calculate the number of transducer elements of the ultrasound array that are receiving the pulse train as an echo of the ultrasound pulse and the number that are not.

As the number of transducer elements used in the transducer array of an ultrasound system increases, the reduction in data rate for data being transmitted by the ultrasound array becomes increasingly significant, as a smaller percentage of the transducer elements of the transducer array are active at the same time. Imaging with an array of ultrasonic transducer elements is usually carried out at F numbers less than 1, as there are physical limitations in the physics of propagation of waves from transducer elements. This is usually done by reducing the number of elements of the array that are active at any given time. The F number may be the ratio of focal length to aperture of the transducer array.

For example, an event-based ultrasound system may include a transducer array with 20,000 transducer elements, may use 50e-9s time stamps, a 100 ms global sync pulse and 1 bit for storing the up-down value for an event, requiring 36 bits to store a single event from a single transducer element based on the electrical signal output by the transducer event. The final data rate of the transducer array in such an ultrasound system, assuming that 10% of the transducer elements of the transducer array are active at any given time, would be 1.4 Terabits per second. A conventional ultrasound system with a transducer array of the same size, not using an event-based system, would have a data rate of 5.6 Terabits per second. The data rate may be variable with number of transducer elements, system time reset, and sampling rate, all of which may be adjusted for quality or cost of electronics. Additionally, the data from the event-based ultrasound system need not be read out serially, as instead it may be transported over a multi-bit bus realized electronically, optically or even via radio waves, for example, using very high bandwidth short distance radio transceivers that may be able to provide the required bandwidth.

In some implementations, an event-based ultrasound system may use local memory to store data for events on a per transducer element basis or a per sub-group basis. For example, a transducer element may have a local memory that may be able to store data for 100 events, with the data for events stored as a timestamp and a bit for the up-down value. When data for the events are read out from the local memory, the address of the transducer element for which the event was recorded may be added, either prepended or appended, to the data for the events. Alternatively, each group of address-timestamp-up-down value bit for events may be stored in a memory that is local to a group of transducer elements. The use of local memory on the transducer elements may allow the data for events to be read out sometime after the events are recorded, further reducing the potential for congestion or the need for very high bandwidth data channels.

In some implementations, typical frames may be reconstructed on the back end. The data from the transducer elements may be aggregated and sorted into a deep time ordered matrix of the data points. Once this is assembled, post processing may be used to reconstruct the waveforms at a target quantization level and sampling frequency. The size, and cost, of the system may be shifted from the transducer array to the back-end system where space, power, and cooling may be more available. Capturing data for later reconstruction may obviate the need for real time imaging.

In some implementations, the raw events may be used to process the data. For example, convolution with linear kernels, for example, Gaussian, Sobel, or similar, in the address event (spike) domain may produce results without having to reconstruct the frame. This may save computational time. As the underlying nature of the data being sent may be events, or spikes, the data may be directly input to neural network or other spike based analysis framework.

In some implementations, events may be sent to be stored in any suitable storage system, such as, for example, a server system, and may be retrieved a later time for image reconstruction by any suitable computing device or system.

An event-based ultrasound system may allow transducer and system designers wider latitude in creating ultrasound system. Receive beamforming on high element count matrix transducers may allow for the avoidance of the need for microbeamforming, cable thickness may be reduced for the same number of elements in a transducer array, or the number of transducer elements per transducer array for a given cable size may be increased while still reducing data transmission, storage, and computational needs, and lowering thermal/power requirements in the handle.

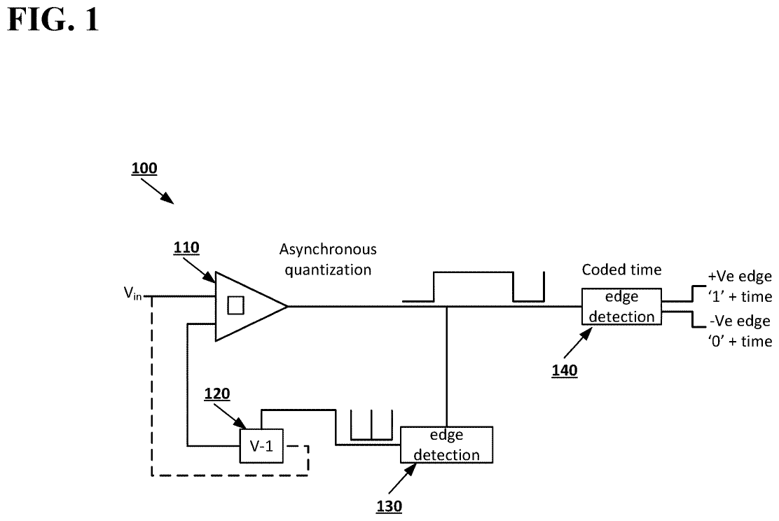

FIG. 1 shows an example representation of a circuit for an event-based ultrasound system. The event-detection circuit 100 may be connected to a transducer element and may receive an electrical signal output by the transducer element as input voltage Vᵢₙ to the non-inverting input of an operational amplifier/comparator 110. The input to the inverting input of the operational amplifier/comparator 110 may also be Vᵢₙ with a time delay introduced by a time delay component 120. The time delay component 120 may, for example, receive as input Vᵢₙ₀ at a time t=0, and then output Vᵢₙ₍₀₎ to the inverted input of the operational amplifier/comparator 110 at a time t=1 when the operational amplifier/comparator 110 is also receiving as input at the non-inverted input a voltage Vᵢₙ₍₁₎. The time delay component 120 takes a clock signal from the edge detection component 130, which may detect when there is an edge-up or edge-down in the output of the operational amplifier/comparator. This clock signal discards the current stored value and shifts the current Vᵢₙ to memory and applies it at the non-inverting input of the operational amplifier/comparator 110. The output of the operational amplifier/comparator 110 may be input to a time stamping edge detection component 140, which may determine if the signal includes an edge-up, edge-down, or neither. On detection of an edge-up, the time stamping edge detection component 140 may generate an up-down value of “1” and a timestamp, both of which may be stored as an event. On detection of an edge-down, the time stamping edge detection component 140 may generate an up-down value of “0” and a timestamp, both of which may be stored as an event. The detection of edge-up or edge-down may include subjecting the signal to a noise threshold and may also use time gain compensation to account for attenuation of the signal over time. When the time stamping edge detection component 140 detects no edge-up or edge-down in the electrical signal input to it, the time stamping edge detection component 140 may not generate and store a timestamped bit as an event, as no event may have occurred. The events may be stored in any suitable memory, including memory local to the transducer element connected to the event-detection circuit 100, memory that is local to a group of transducer elements, memory local to an entire transducer array, or memory that is part of a computing device that may be connected to the event-detection circuit 100 in any suitable manner, including through any suitable wired or wireless connection. The time stamping edge detection component 140 may also store as part of an event an address of the transducer element connected to the time stamping edge detection component 140. The event-detection circuit 100 may act a single-bit quantizer for the electrical signal output by the transducer element it is connected to. Implicit in the design of the event-detection circuit 100, or quantization circuit, is the ability to only send data when change occurs. In conventional ultrasound systems, quiet periods in which no changes are occurring would have to be explicitly defined so as not to gather and send data.

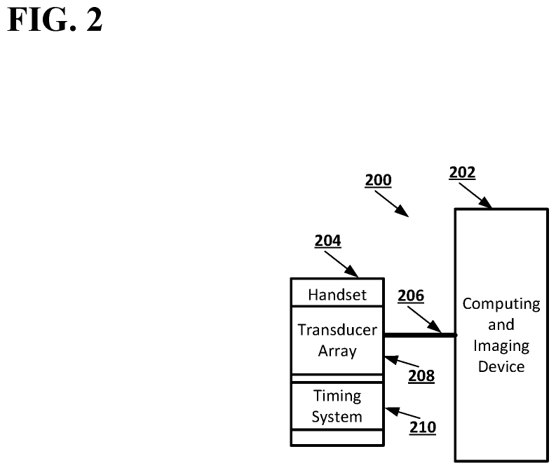

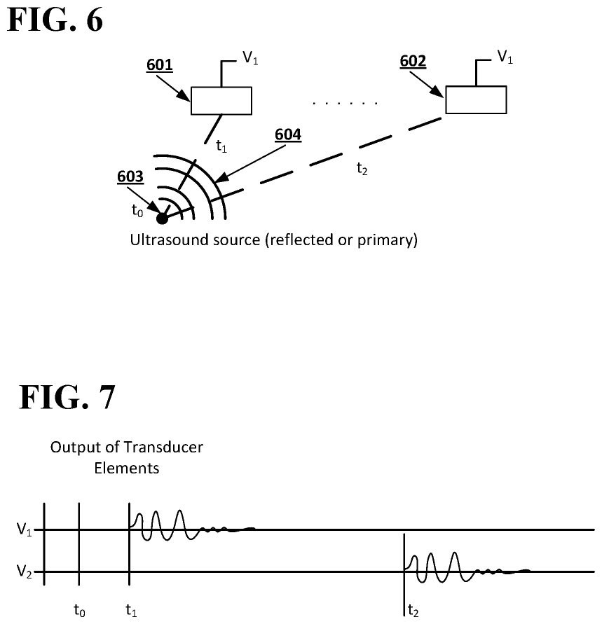

FIG. 2 shows an example system for an event-based ultrasound system. An ultrasound system 200 may include a handset 204 and a computing and imaging device 202 connected in any suitable manner, such as by a connection 206, to allow for data to be transmitted in both directions between the handset 204 and the computing and imaging device 202. The handset 204 may include a transducer array 208 that may include ultrasonic transducer elements arranged in an array and a timing system 210 that may ensure consistency in timestamping data for events across the transducer elements of the transducer array 208. The computing and imaging device 202 may include any suitable computing hardware, running any suitable software, and any other suitable electronics to operate the ultrasound system 200, including supplying power and control signals to ultrasonic transducer elements of the transducer array 208, for example, through the connection 206, receiving signals from the transducer elements of the transducer array 208, performing any suitable computation to generate images from the signals received from the transducer elements of the transducer array 208, and displaying generated images, for example, on a display directly connection to the computing and imaging device 202, or otherwise sending the generated images to a device, for example, a tablet or phone, that can display the generated images. The computing and imaging device 202 may have any suitable interface to allow a user to control the ultrasound system 200. The computing and imaging device 202 may be, or may include, a computer 20 as shown in in FIG. 8. The connection 206 may be any suitable connection for carrying data between the handset 204 and the computing and imaging device 202. The connection 206 may be a wired connection, for example, a cable connecting the handset 204 to the computing and imaging device 202 directly, or a wireless connection, for example, using any suitable radios and wireless communication protocols. In some implementations, the connection 206 may incorporate any suitable mixture of wired and wireless connections across any suitable communications networks that may include, for example, the Internet, which may allow the handset 204 to be remote from the computing and imaging device 202.

The transducer array 208 may be able to generate pulses of ultrasonic waves and then detect echoes of the ultrasonic waves 120 as they are reflected. When a transducer element of the transducer array 208 receives an echo of a pulse, the transducer element may generate a voltage with an amplitude and frequency that corresponds to the received echo and may be output as an electrical signal from the transducer element to an event-detection circuit such as the event-detection circuit 100. The event-detection circuit 100 may determine if the electrical signal output from the transducer element includes an edge-up or edge-down event for the transducer element, and if so, may generate and store data for the event including an up-down value, timestamp, and address of the transducer element. Timestamps for all events generated by the transducer elements of the transducer array 208 may be based on the same clock that may be part of the timing system 210. The data for the event may be stored in memory on the handset 204, for example, memory local to the transducer element, a group of transducer elements, or the entire handset 204, and later transmitted across the connection 206 to the computing and imaging device 202. The data for the event may also be transmitted directly from the event-detection circuit to computing and imaging device 202 across the connection 206 in real-time. The computing and imaging device 202 may use data for events from the handset 204 received in real-time to generate images in real-time or may store the data for use at a later time. The computing and imaging device 202 may use data for events from the handset 204 received not in real-time to generate images from the data as it is received, or

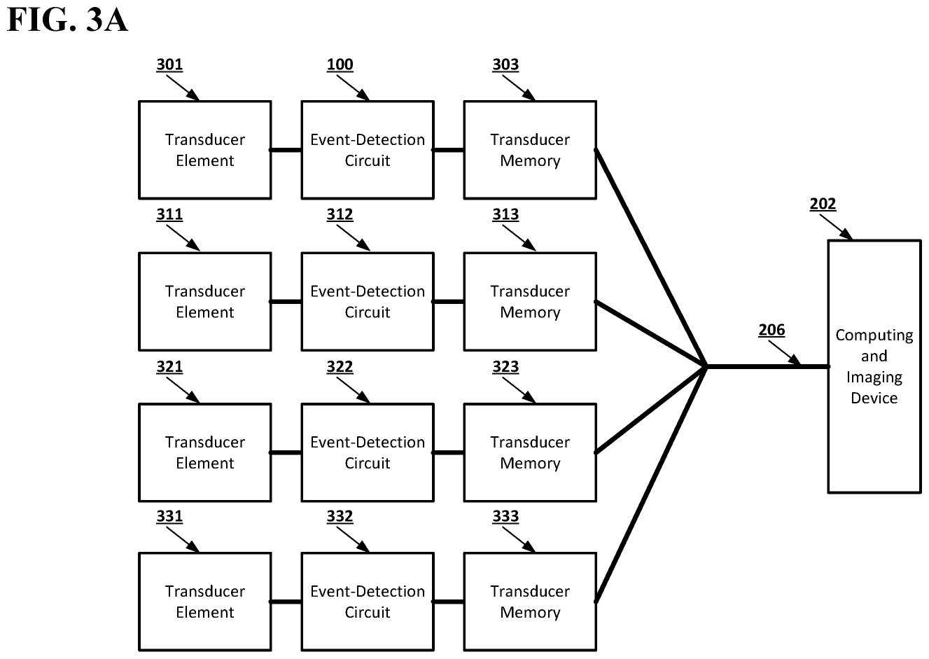

FIG. 3A shows an example arrangement for an event-based ultrasound system. In some implementations, each transducer element of the transducer array 208 may have local memory. For example, a transducer element 301 of the transducer array 208 may be connected to the event-detection circuit 100 which may output data for events to transducer memory 303, which may be any suitable form of volatile or non-volatile memory of any suitable storage capacity. The transducer memory 303 may be physically located proximate to the transducer element 301, for example, sharing a circuit board with other control electronics for the transducer element 301. A transducer element 311 of the transducer array 208 may be connected to an event-detection circuit 312 which may output data for events to transducer memory 313 which may be separate from the transducer memory 313. Similarly, transducer elements 321 and 322 may be connected to, respectively, event-detection circuits 322 and 332 which may output data to, respectively, transducer memory 323 and 333, which may be separate from each other and from the transducer memories 303 and 313. The transducer memories 303, 313, 323, and 333 may store data from events for their respective transducer elements 301, 311, 321, and 331 for any suitable amount of time, and the data for the events may be transmitted across the connection 206 to the computing and imaging device 202 at any suitable time. For example, data for events may be read out from the transducer memories 303, 313, 323, and 333 and transmitted across the connection 206 at regular intervals or based on the occurrence of any suitable event, including, for example, based on the number events for which data is stored in the transducer memories 303, 313, 323, and 333. Data for events stored in the transducer memories 303, 313, 323, and 333 may be stored with only the up-down value and timestamp, with the address of the transducer element that generated the event being added only when the data for the events are read out from the transducer memories 303, 313, 323 and 333 to be transmitted across the connection 206.

FIG. 3B shows an example arrangement for an event-based ultrasound system. In some implementations, groups of transducer elements of the transducer array 208 may have local group memory. For example, the transducer elements 301, 311, 321, and 331 may share transducer group memory 390, which may be any suitable form of volatile or non-volatile memory of any suitable storage capacity. The transducer group memory 390 may store data for events from all of the transducer elements in the group that includes the transducer elements 301, 311, 321, and 331. The data for events may be stored for any suitable amount of time, and the data for the events may be transmitted across the connection 206 to the computing and imaging device 202 at any suitable time. For example, data for events may be read out from the transducer group memory 390 and transmitted across the connection 206 at regular intervals or based on the occurrence of any suitable event, including, for example, based on the number events for which data is stored in the transducer group memory 390. Data for events stored in the transducer group memory 390 may include the up-down value, timestamp, and address of the transducer element that generated each of the events.

FIG. 3C shows an example arrangement for an event-based ultrasound system. In some implementations, the transducer elements of the transducer array 208 may send data for events directly to the computing and imaging device 202 without storing them in memory local to the transducer array 208. For example, the event-detection circuits 100, 312, 322, and 332 may for transducer elements 301, 311, 321, and 331 may be connected directly to the connector 206. Data for events generated based on electrical signals from the transducer elements 301, 311, 321, and 331, and from all other transducer elements of the transducer array 208, may be transmitted to may be transmitted across the connection 206 to the computing and imaging device 202 as soon as they are generated, and may be stored in memory local to the computing and imaging device 202. Data for events transmitted to the computing and imaging device 202 may include the up-down value, timestamp, and address of the transducer element that generated each of the events.

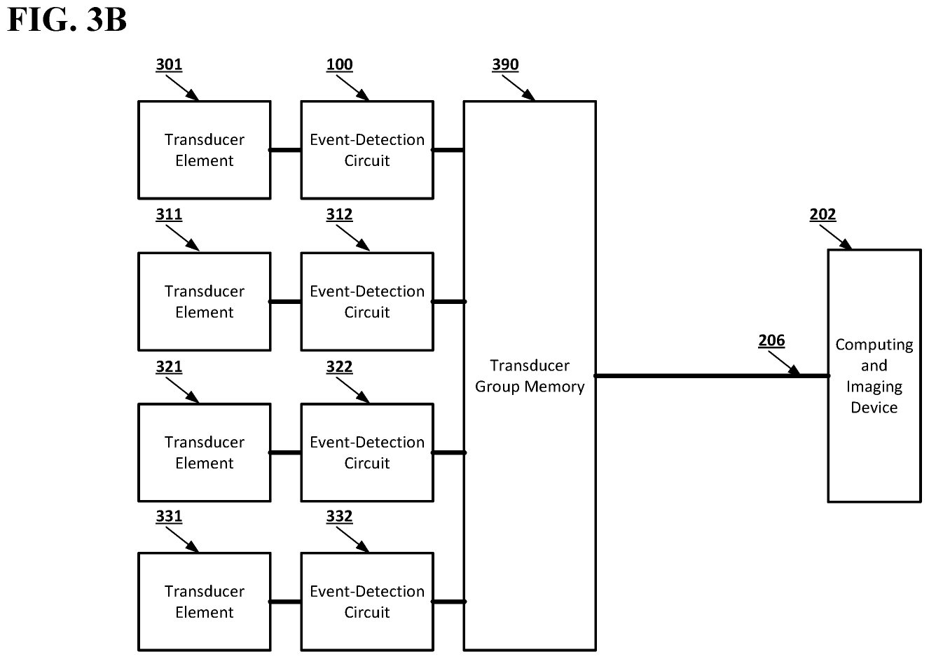

FIG. 4A shows an example arrangement for an event-based ultrasound system. The computing and imaging device 202 may receive data 401 including data for events from the transducer array 208, for example, over the connection 206. The data for an event may be in the format [up-down value; time; transducer element address]. The data 401 may include data for a single event that may have an up-down value of “1”, may have occurred at a time of “1”, and may have been generated based on the electrical signal from a transducer element at address (0,0) in the transducer array 208. The data for the event in the data 401 may have been generated any amount of time prior to being sent to the computing and imaging device 202. The computing and imaging device 202 may use the data for the event from the data 401 to generate an image 441, which may be a frame representing what the transducer array 208 sees at the time t=1. The transducer array 208 may include four transducer elements arranged in a grid, and the image 441 may include four pixels, 411, 412, 413, and 414, which each represent one of the four transducer elements of the transducer array 208. The data for the event in the data 401 may indicate that the transducer element located at address (0,0) experienced an edge-up event at the time t=1, so the computing and imaging device 202 may generate the image 441 to show the pixel 411 representing the transducer element at (0,0) to be “on” while the pixels 412, 413, and 414 are “off”.

FIG. 4B shows an example arrangement for an event-based ultrasound system. The computing and imaging device 202 may receive data 402 including data for events from the transducer array 208, for example, over the connection 206. The data 402 may include data for three events. The first event may have an up-down value of “1”, may have occurred at a time of “2”, and may have been generated based on the electrical signal from a transducer element at address (0,1) in the transducer array 208. The second event may have an up-down value of “1”, may have occurred at a time of “2”, and may have been generated based on the electrical signal from a transducer element at address (1,0) in the transducer array 208. The third event may have an up-down value of “0”, may have occurred at a time of “4”, and may have been generated based on the electrical signal from the transducer element at address (0,0) in the transducer array 208. The data for the events in the data 402 may have been generated any amount of time prior to being sent to the computing and imaging device 202 and after the data for the event in the data 401 was generated.

The computing and imaging device 202 may use the data for the events from the data 402 to generate images 441, 442, 443, and 444, which may be frames representing what the transducer array 208 sees at the times t=2, t=3, and t=4 respectively. The data for the first event in the data 403 may indicate that the transducer element located at address (0,1) experienced an edge-up event at the time t=2 and data for the second event in the data 403 may indicate that the transducer element located at address (1,0) experienced an edge-up event at time t=2. The computing and imaging device 202 may generate the image 442 to show the pixels 412 and 413, representing the transducer elements at (0,1) and (1,0) to be “on”, the pixel 411 remaining “on” as there was no event showing the transducer element at address (0,0) experienced an edge-down event, and pixel 414 remaining “off”.

The data 402 may include no events with a timestamp of t=3, indicating no edge-up or edge-down were experienced at any of the transducer elements 411, 412, and 413 after time t=2 and before time t=4. The computing and imaging device 202 may generate the image 443 to show no changes from the image 442.

The data for the third event in the data 402 may indicate that the transducer element located at address (0,0) experienced an edge-down event at the time t=4. The computing and imaging device 202 may generate the image 444 to show the pixels 412 and 413 remaining “on” as no edge-down event for the transducers at (0,1) and (1,0) may have occurred at time t=3 or time t=4, the pixel 411 representing the transducer element at (0,0) switched from “on” to “off” based on the edge-down even in the data 402, and the pixel 414 remaining “off”. In this way, the computing and imaging device 202 may be able to generate images using data from the transducer elements of the transducer array 208 based on events that occur at the transducer elements, are detected and timestamped using event-detection circuits, are stored with addresses, and are sent to the computing and imaging device 202 either in real-time or at intervals, without requiring that the computing and imaging device 202 receive data about the state of each transducer element and every time step. This may result in a reduction of the amount of data sent to the computing and imaging device 202 while still allowing the computing and imaging device 202 to generate images based on the ultrasonic imaging performed by the transducer array 208.

In this exemplary implementation, data for events may be stored in memory local to the transducer array 208 before being transmitted to the computing and imaging device 202, for example, at regular intervals. This may result in the computing and imaging device 202 receiving data for events with different timestamps at the same time. In an implementation where the computing and imaging device 202 receives data for events in real-time, as the events are generated, the computing and imaging device 202 may not receive data for events with different timestamps at the same time.

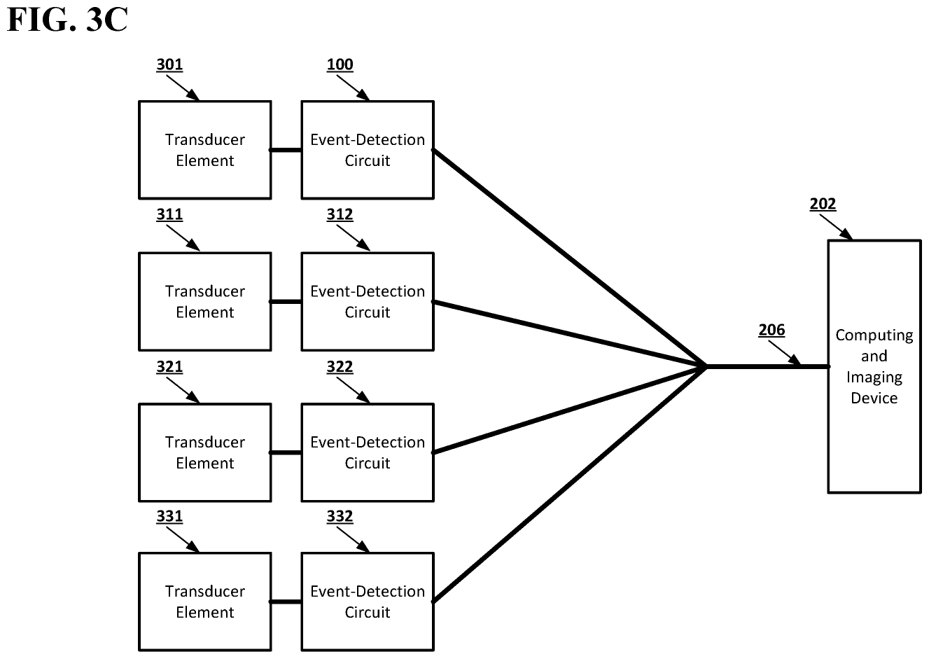

FIG. 5 shows an example procedure for an event-based ultrasound system. At 500, an edge-up or edge-down event may be detected. For example, the event-detection circuit 100 may detect an edge-up or edge-down event in the electrical signal output by the transducer element 301 based on the receiving of the echo of an ultrasonic pulse by the transducer element 301. The edge-up or edge-down event may be detected based on a change in the electrical signal output by the transducer element 301, for example, an increase or decrease in the voltage of the electrical signal.

At 502, an up-down value may be set for the event. For example, the event-detection circuit 100 may set the up-down value for the event to “1” is the event was an edge-up event, and to “0” if the event was an edge-down event. The up-down value may be stored as a single bit.

At 504, the event may be timestamps. For example, the timing system 210 may provide a clock that is consistent across all transducers in the transducer array 208, and may be used to generate a timestamp for the event at the time the event is detected by the event-detection circuit 100. The timestamp for the event may be in any suitable format, and may be stored using any suitable number of bits, for example, 25 bits.

At 506, an address may be added to the event. For example, the address of the transducer element 301 may be added to the event along with the up-down value and the timestamp. The address may identify the location of the transducer element 301 within the transducer array 208. The address may be added when the event is generated or may be added at a later time, for example, after the event is read out from local memory, for example, the transducer memory 303 and before the event is transmitted to the computing and imaging device 202. The address may be added to the event by any suitable electronic component of the transducer array 208, including, for example, the timing system 210, the event-detection circuit 100, or another component of the control electronics for the transducer element 301.

At 506, the event may be transmitted. For example, if the data for the event was stored in the transducer memory 303, the data for the event may be read out, have an address added as in 506, and transmitted to the computing and imaging device 202 through the connection 206, for example, as data 401. Any other data for events stored in the transducer memory 303 may be transmitted at the same time. If the data for the event was stored in the transducer group memory 390, the data for the event may be read out and transmitted to the to the computing and imaging device 202 through the connection 206 along with data for events for other transducer elements that may be stored in the transducer group memory 390. If data for events is not stored in memory local to the transducer array 208, the data for the event may be transmitted to the computing and imaging device 202 through the connection 206 in real-time, for example, as soon as it is generated.

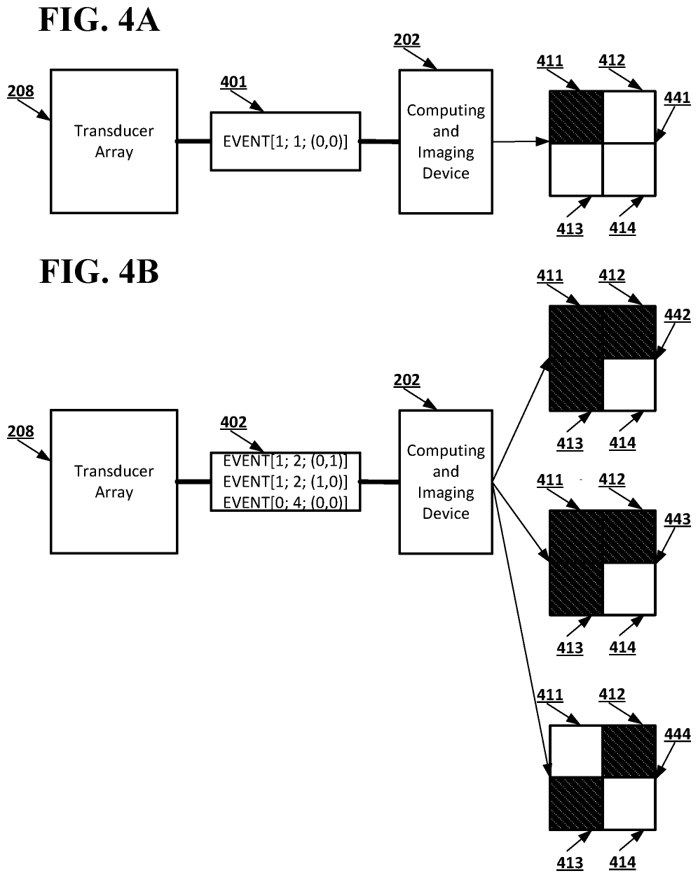

FIG. 6 shows an example representation of data collection by an ultrasound system. For example, transducers elements 601 and 602 may receive ultrasonic waves 604 that may be generated by or reflected off of a source 603. The transducer element 601 may receive the ultrasonic waves 604 at a time t₁, while the transducer element 602 may receive the ultrasonic waves at a time t₂ subsequent to time t₁.

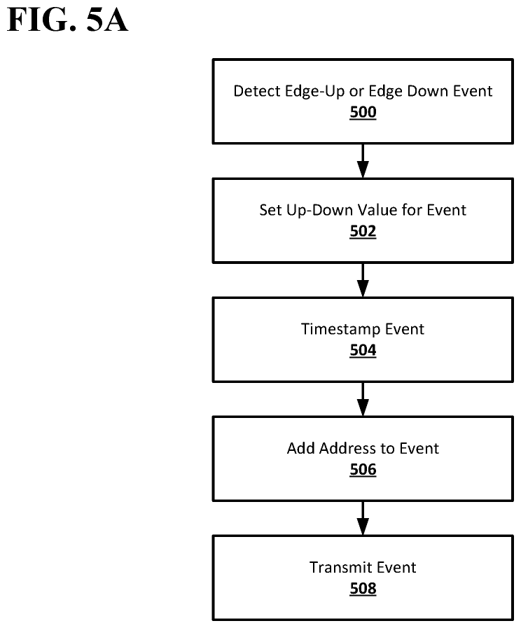

FIG. 7 shows an example representation of data collection by an ultrasound system. In a conventional ultrasound system, data may be collected from transducer elements 601 and 602 starting at a time t₀ to past a time t₂. This may result in a large amount of unnecessary data being collected. In an event-based ultrasound system, data may be generated around the times represented by t₁ and t₂, which may be when the ultrasonic waves 604 arrive at the transducer elements 601 and 602, respectively, resulting in increases in the voltage V₁ at a time t₁ and increases in the voltage V₂ at time t₂. Events may be generated around t₁ and t₂, for example initial edge-up events and later edge-down events, and these events may be sent back to the computing and imaging device 202. The rest of the time, no data may be sent to the computing and imaging device 202, as no events may be generated.

In a conventional ultrasound system, the data between the occurrence of events is usually subject to comparison to upper and lower thresholds to produce cleaner images. This may effectively throw away the data between events because it carries no useful information. The event-based ultrasound system also throws away this data but does so earlier in the signal processing chain than conventional ultrasound systems, so that this data does not to be transmitted across the connection 206 before being discarded, reducing the bandwidth needed across the connection 206.

Thresholding may also take into account attenuation of signals over time, for example, using Time Gain Compensation (TGC), which may apply a constant gain for estimated travel distance, for example, 0.5 dB/cm/MHz at 1540 m/s.

For example, Table 1 shows data for convention and event-based ultrasound systems used with a high end ultrasound system operating at 20 MHz sample rate with a transducer of 128 elements, where the event-based ultrasound system only has 25% of the transducer elements active, as in reporting a change in signal received at the transducer element, at any given point in time.

TABLE 1

| | | Time | ADC | Number of | | | |
|---|---|---|---|---|---|---|---|
| | Sampling | stamp | Num | Transducer | Address | Data | |
| | Frequency | bits | Bits | Elements | bits | Rate/sec. | |
| Conventional | 20 MHz | N/A | 14 | 128 | n/a | 35.8 | Gbits/sec |
| Event-Based | 50 nS time | 25 | N/A | 128 | 7 | 20 | G bits/sec |
| | stamp | | | | | | |

Table 1 assumes that the clock is reset every 1 second, requiring 25 bits to store a single time stamp. Reducing the global reset/sync time to 100 ms may reduce the bit count for storing a single time stamp to 21 bits, reducing the overall data rate to 18 Gbps.

In some implementations, data for events may be converted during post processing to a “standard” regularly sampled dataset to allow for ingestion by legacy systems.

Implementations of the presently disclosed subject matter may be implemented in and used with a variety of component and network architectures. FIG. 8 is an example computer 20 suitable for implementations of the presently disclosed subject matter. The computer 20 includes a bus 21 which interconnects major components of the computer 20, such as a central processor 24, a memory 27 (typically RAM, but which may also include ROM, flash RAM, or the like), an input/output controller 28, a user display 22, such as a display screen via a display adapter, a user input interface 26, which may include one or more controllers and associated user input devices such as a keyboard, mouse, and the like, and may be closely coupled to the I/O controller 28, fixed storage 23, such as a hard drive, flash storage, Fibre Channel network, SAN device, SCSI device, and the like, and a removable media component 25 operative to control and receive an optical disk, flash drive, and the like.

The bus 21 allows data communication between the central processor 24 and the memory 27, which may include read-only memory (ROM) or flash memory (neither shown), and random access memory (RAM) (not shown), as previously noted. The RAM is generally the main memory into which the operating system and application programs are loaded. The ROM or flash memory can contain, among other code, the Basic Input-Output system (BIOS) which controls basic hardware operation such as the interaction with peripheral components. Applications resident with the computer 20 are generally stored on and accessed via a computer readable medium, such as a hard disk drive (e.g., fixed storage 23), an optical drive, floppy disk, or other storage medium 25.

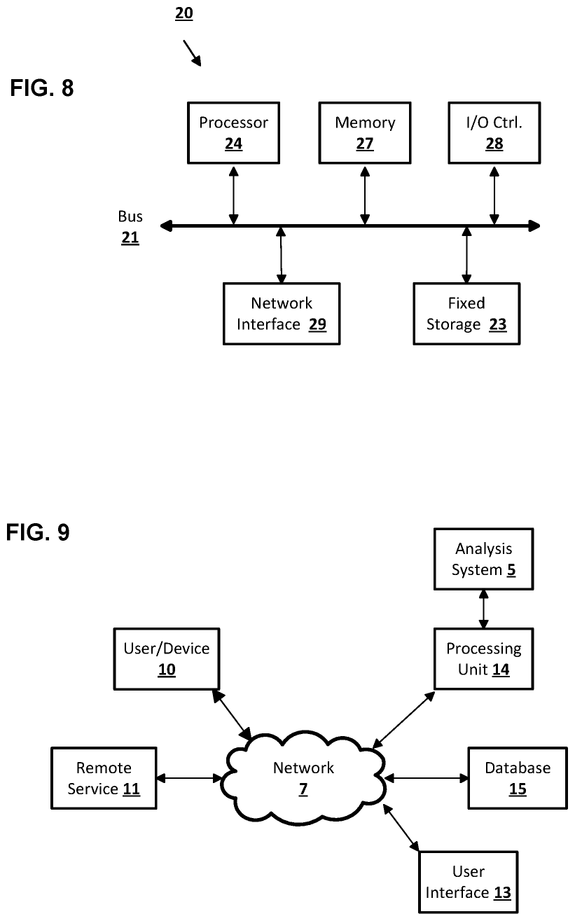

The fixed storage 23 may be integral with the computer 20 or may be separate and accessed through other interfaces. A network interface 29 may provide a direct connection to a remote server via a telephone link, to the Internet via an internet service provider (ISP), or a direct connection to a remote server via a direct network link to the Internet via a POP (point of presence) or other technique. The network interface 29 may provide such connection using wireless techniques, including digital cellular telephone connection, Cellular Digital Packet Data (CDPD) connection, digital satellite data connection, or the like. For example, the network interface 29 may allow the computer to communicate with other computers via one or more local, wide-area, or other networks, as shown in FIG. 9.

Many other devices or components (not shown) may be connected in a similar manner (e.g., document scanners, digital cameras, and so on). Conversely, all of the components shown in FIG. 8 need not be present to practice the present disclosure. The components can be interconnected in different ways from that shown. The operation of a computer such as that shown in FIG. 8 is readily known in the art and is not discussed in detail in this application. Code to implement the present disclosure can be stored in computer-readable storage media such as one or more of the memory 27, fixed storage 23, removable media 25, or on a remote storage location.

FIG. 9 shows an example network arrangement according to an implementation of the disclosed subject matter. One or more clients 10, 11, such as local computers, smart phones, tablet computing devices, and the like may connect to other devices via one or more networks 7. The network may be a local network, wide-area network, the Internet, or any other suitable communication network or networks, and may be implemented on any suitable platform including wired and/or wireless networks. The clients may communicate with one or more servers 13 and/or databases 15. The devices may be directly accessible by the clients 10, 11, or one or more other devices may provide intermediary access such as where a server 13 provides access to resources stored in a database 15. The clients 10, 11 also may access remote platforms 17 or services provided by remote platforms 17 such as cloud computing arrangements and services. The remote platform 17 may include one or more servers 13 and/or databases 15.

More generally, various implementations of the presently disclosed subject matter may include or be implemented in the form of computer-implemented processes and apparatuses for practicing those processes. The disclosed subject matter also may be implemented in the form of a computer program product having computer program code containing instructions implemented in non-transitory and/or tangible media, such as floppy diskettes, CD-ROMs, hard drives, USB (universal serial bus) drives, or any other machine readable storage medium, wherein, when the computer program code is loaded into and executed by a computer, the computer becomes an apparatus for practicing implementations of the disclosed subject matter. Implementations also may be implemented in the form of computer program code, for example, whether stored in a storage medium, loaded into and/or executed by a computer, or transmitted over some transmission medium, such as over electrical wiring or cabling, through fiber optics, or via electromagnetic radiation, wherein when the computer program code is loaded into and executed by a computer, the computer becomes an apparatus for practicing implementations of the disclosed subject matter. When implemented on a general-purpose microprocessor, the computer program code segments configure the microprocessor to create specific logic circuits. In some configurations, a set of computer-readable instructions stored on a computer-readable storage medium may be implemented by a general-purpose processor, which may transform the general-purpose processor or a device containing the general-purpose processor into a special-purpose device configured to implement or carry out the instructions.

Implementations may use hardware that includes a processor, such as a general-purpose microprocessor and/or an Application Specific Integrated Circuit (ASIC) that embodies all or part of the techniques according to embodiments of the disclosed subject matter in hardware and/or firmware. The processor may be coupled to memory, such as RAM, ROM, flash memory, a hard disk or any other device capable of storing electronic information. The memory may store instructions adapted to be executed by the processor to perform the techniques according to embodiments of the disclosed subject matter.

The foregoing description, for purpose of explanation, has been described with reference to specific implementations. However, the illustrative discussions above are not intended to be exhaustive or to limit implementations of the disclosed subject matter to the precise forms disclosed. Many modifications and variations are possible in view of the above teachings. The implementations were chosen and described in order to explain the principles of implementations of the disclosed subject matter and their practical applications, to thereby enable others skilled in the art to utilize those implementations as well as various implementations with various modifications as may be suited to the particular use contemplated.

1. An event-based ultrasound system comprising:
a transducer array comprising transducer elements and a timing system, wherein the transducer elements are connected to event-detection circuits, and wherein the event-detection circuits generate events based on the electrical signals of the transducer elements and wherein data for an event comprises an up-down value, a timestamp based on the timing system, and an address of a transducer element;
a computing and imaging device; and
a connection connecting the transducer array to the computing and imaging device, wherein the data for the events generated by the event-detection circuits is transmitted to the computing and imaging device across the connection.

2. The system of claim 1, wherein an event-detection circuit of the event-detection circuits generates an event when an edge-up or edge-down is detected in an electrical signal from a one of the transducer elements to which the event-detection circuit is connected.

3. The system of claim 2, wherein the event-detection circuit does not generate an event when no edge-up or edge-down is detected in the electrical signal from a one of the transducer elements to which the event-detection circuit is connected.

4. The system of claim 1, further comprising transducer memories connected to the transducer elements, wherein each one of the transducer memories stores data for events generated by an event-detection circuit to which the one of the transducer memories is connected.

5. The system of claim 4, wherein the transducer memories store the data for events without addresses of transducer elements, and wherein the addresses for the transducer elements are added to the data for the events when the data for the events is read out from the transducer memories.

6. The system of claim 1, further comprising transducer group memories connected to transducer elements, wherein each of the transducer group memories stores data for events generated by a group of event-detection circuits to which the one of the transducer group memories is connected.

7. The system of claim 1, wherein the up-down value for an event comprises a single bit generated by one of the event-detection circuits.

8. The system of claim 1, wherein data for the events generated by the event-detection circuits is transmitted to the computing and image device using the connection in real time.

9. The system of claim 1, wherein the data for the events generated by the event-detection circuits is transmitted to the computing and image device using the connection at intervals.

10. The system of claim 1, wherein the timing system comprises a clock, and wherein all timestamps for all events generated by the event-detection circuits are based on the clock of the timing system.

11. The system of claim 1, wherein the computing and imaging device generates images from the data for the events received over the connection.

12. A method for an event-based ultrasound system comprising:
detecting, by an event-detection circuit an event, wherein the event comprises an edge-up event or edge-down event in an electrical signal input to the event-detection circuit from a transducer element;
setting, by the event-detection circuit, an up-down value for data for the event based on which of an edge-up event and an edge-down event was detected;
timestamping the event by generating a timestamp that indicates a time at which the event occurred for data for the event;
adding an address of the transducer element to the data for the event; and
transmitting the data for the event to a computing device.

13. The method of claim 12, further comprising storing the up-down value and timestamp of the data for the event are in a transducer memory that is local to the transducer element before adding the address of the transducer element to the data for the event; and
reading out the up-down value and the timestamp of the data for the event from the transducer memory before adding the address of the transducer element to the data for the event.

14. The method of claim 12, further comprising storing the data for the event comprising the up-down value, timestamp, and address in a transducer group memory that is local to a group of transducer elements.

15. The method of claim 12, wherein timestamping the event by generating a timestamp that indicates a time at which the event occurred for data for the event comprises uses a time of a clock of a timing system.

16. The method of claim 12, further comprising not transmitting data based on the electrical signal output by the transducer element to the computing device in absence of data for an event generated after detection of the event by the event-detection circuit.

17. The method of claim 12, further comprising:
receiving, by the computing device over a connection, the data for the event; and
generating, by the computing device, and image based on the data for the event and data for one or more other events, wherein the one or more other events occurred at any time relative to the event.

18. The method of claim 12, where the edge-detection circuit is a single-bit quantizer for the electrical signal.

19. The method of claim 12, wherein the address of the transducer element indicates the location of the transducer element within a transducer array.

20. The method of claim 12, wherein an edge-up event occurs when the transducer element transitions from generating a voltage that is below a noise floor to generating a voltage that is above the noise floor, and wherein an edge-down event occurs when the transducer element transitions from generating a voltage that is above the noise to floor to generating a voltage that is below the noise floor.

21. The method of claim 12, wherein time gain compensation is used to account for attenuation of electrical signals generated by the transducer elements over time.

22. The method of claim 12, further comprising converting data for events comprising at least the data for the event to a regularly sampled dataset.
